(19) United States
(12) Patent Application Publication
(10) Pub. No.: US 2022/0323172 A1
(43) Pub. Date: Oct. 13, 2022

(54) SURGICAL SPONGE ACCOUNTING SYSTEM AND METHOD OF USE

(71) Applicant: Innovation Ventures IP at UKHS, LLC, Westwood, KS (US)

(72) Inventor: Lorie Hassel-Chuang, Westwood, KS (US)

(21) Appl. No.: 17/717,593

(22) Filed: Apr. 11, 2022

Related U.S. Application Data
(60) Provisional application No. 63/172,894, filed on Apr. 9, 2021.

(51) Int. Cl. A61B 50/30

(52) U.S. Cl. CPC A61B 50/30; A61B 2090/0804

(57) ABSTRACT

The present invention relates to a novel surgical sponge accounting system and the method of use. In particular, the invention relates to a surgical sponge accounting system that dispenses surgical sponge counter bags, organizes the bags and then allows for used sponges to be placed in the sponge counter bags to clearly display and aide in easily accounting for all surgical sponges used during a procedure.

### CROSS REFERENCE TO RELATED APPLICATION

This application claims the benefit of the U.S. Provisional Application Ser. No. 63/172,894, entitled “Surgical Sponge Accounting System and Method of Use,” filed Apr. 9, 2021, the contents of which are incorporated by reference herein, for all purposes, in its entirety.

### FIELD OF THE INVENTION

The present invention relates to a novel surgical sponge accounting system that includes a surgical sponge bag dispenser and used surgical sponge holder device and the method of use. In particular, the invention relates to a surgical sponge accounting system that includes a surgical sponge counter bag dispenser and used sponge accounting holder device that dispenses surgical sponge counter bags, organizes the bags for a visual review and then allows for used sponges to be placed in the sponge counter bags to clearly display and aide in easily accounting for all surgical sponges used during a procedure.

### BACKGROUND

Surgical procedures are conducted daily on patients, during these surgical procedures numerous items are used by the healthcare providers. There are many tools and process used to account for surgical items used during and at the end of the procedure, however, there is no definitive method to account for all the objects and in particular used surgical sponges. It is known that in too many instances sponges, and even instruments, have been left behind inside of a patient after a surgical procedure, due to the item not being accounted for during and at the end of the procedure. Because no standard system is employed universally healthcare providers many times must rely on ad hoc counting to account for the sponges and other surgical items. In the healthcare industry the term Retained Surgical Items (RSI) is a general term that refers to items such as tools, materials or sponges that are inadvertently left in a patient's body during a surgical procedure. The tracking of these RSI events outlines that these events happen minimally, but when an RSI event occurs it creates an increase cost for treatment and in certain cases death of the patient. The health industry as a whole recognizes the severity of RSI events and identifies them as “never events” or further defined as any unanticipated event in a healthcare setting resulting in death or serious physical or psychological injury to a patient or patients, not related to the natural course of the patient's illness. Due to the serious consequences, such events must be reported to regulating agencies for tracking.

Since RSI create numerous issues for both a patient and the healthcare system it is critical that all surgical items, including sponges, are fully accounted for at the conclusion of the surgical procedure to prevent RSI events. However, due to the difficult conditions under which surgical procedures are undertaken, the current systems used for accounting for surgical sponges and other surgical items are not always effective. Despite the use of many systems and devices currently used within the industry for accounting for items, RSI events, especially those due to sponges are still prevalent and a significant issue for patient safety and recovery. The risk of not accurately accounting for surgical sponges during surgical procedures continues to result in RSI and is a significant problem and risk to a patient.

Based on the current state of the industry there exists a need for an accounting system and its method of use that provides an organized and methodical means for disposing of and recording the collection of surgical items and in particular surgical sponges during a surgical procedure. Furthermore, there is a need for a system that improves the efficiency, is ease to use, decreases the duration of counts, offers a centralized counting location, decrease unnecessary movement, promotes safety and decreases human factor errors.

Therefore, it is an object of the present invention to demonstrate an accounting system and method of using a system that provides a surgical sponge counter bag dispenser and a rack system for surgical sponge counter bags that offer a simple, efficient, and reliable method for safely accounting for used surgical sponges or other surgical items used during a surgical procedure.

### SUMMARY OF THE INVENTION

The present invention is directed to a surgical sponge accounting system and the method of use. The invention provides a novel accounting system for surgical staff to dispense surgical sponge counter bags that are hung for visual display on the surgical sponge accounting system. The surgical sponge counter bags are used by the surgical staff to ensure that all used surgical sponges are accounted for after a surgical procedure. The surgical sponge accounting system creates a device that can accommodate a roll of surgical sponge counter bags that is further used for dispensing during a surgical procedure. The device includes a unique housing unit that is configured to hold and dispense the surgical sponge bags. The housing unit includes a view portion to easily view the surgical sponge bag roll. After the surgical sponge bags are dispensed the accounting system provides a counting station for the organization, holding, placement and accounting of used surgical sponges (or other surgical items) within the surgical sponge counter bags and a visible method for accounting for the whereabouts of the used surgical sponges (or other surgical items) during and at the conclusion of the surgical procedure, whereby creating a systematic process to account for surgical sponges and other items.

According to another aspect of the present invention provide a method for accounting for used surgical sponges after a surgical procedure by employing the use of the surgical sponge accounting system to dispense then for the arrangement of the surgical sponge bags to display and account for the used sponges from the surgical procedure. With reference to the figures, the invention is to a novel surgical sponge accounting system and its method of use that includes various embodied principles and concepts that create a system that offers a simple, efficient and reliable method for safely accounting for used surgical sponges and/or other surgical items during and at the conclusion of a surgical procedure.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a first perspective view of the surgical sponge accounting system according to one embodiment.

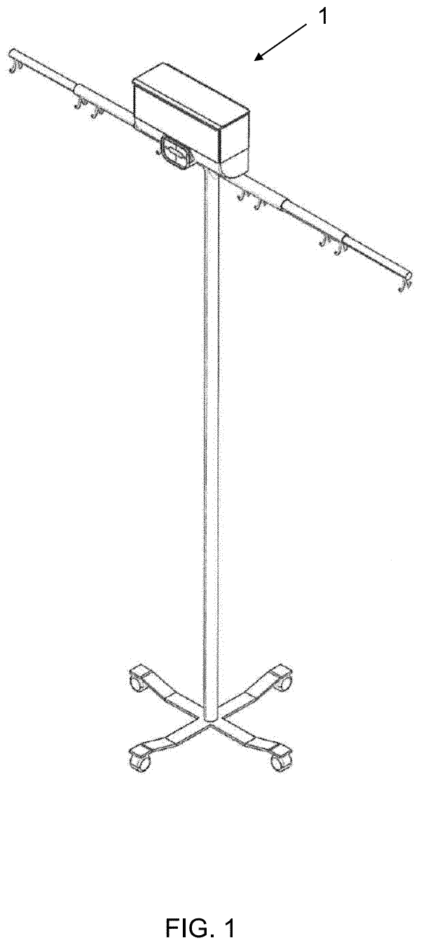

FIG. 2 is a view of the surgical sponge counter bag dispenser for the surgical sponge accounting system according to one embodiment.

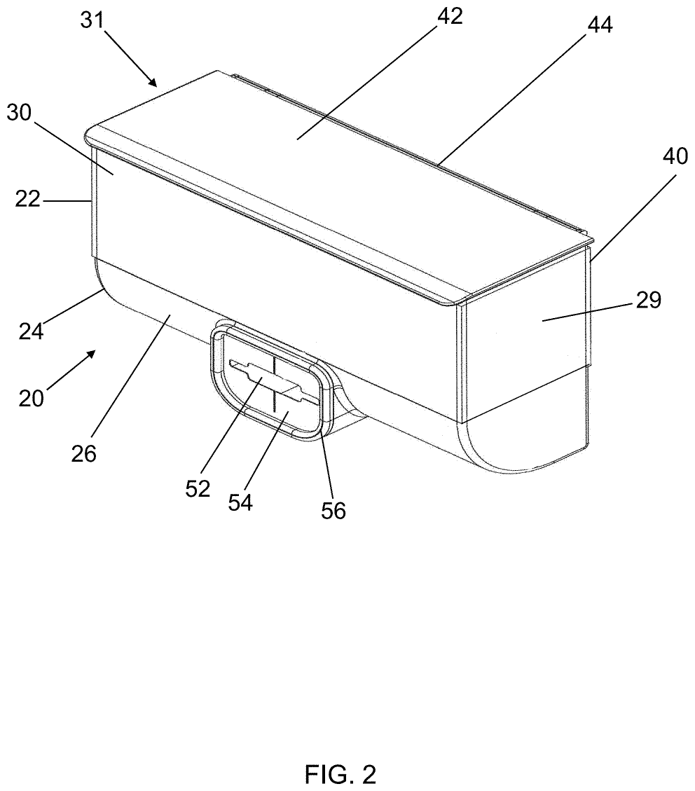

FIG. 3 is a side view of the surgical sponge counter bag dispenser for the surgical sponge accounting system according to one embodiment.

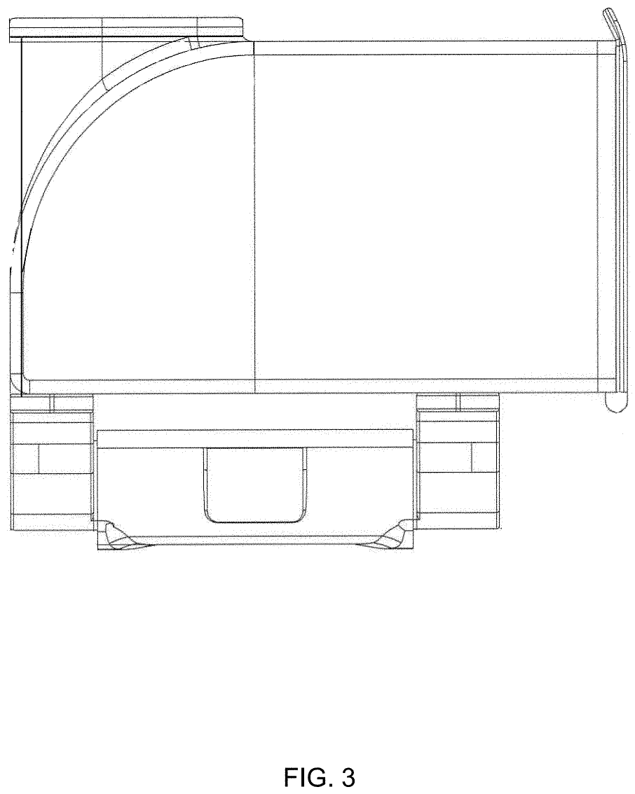

FIG. 4 is a top view of the surgical sponge counter bag dispenser for the surgical sponge accounting system according to one embodiment.

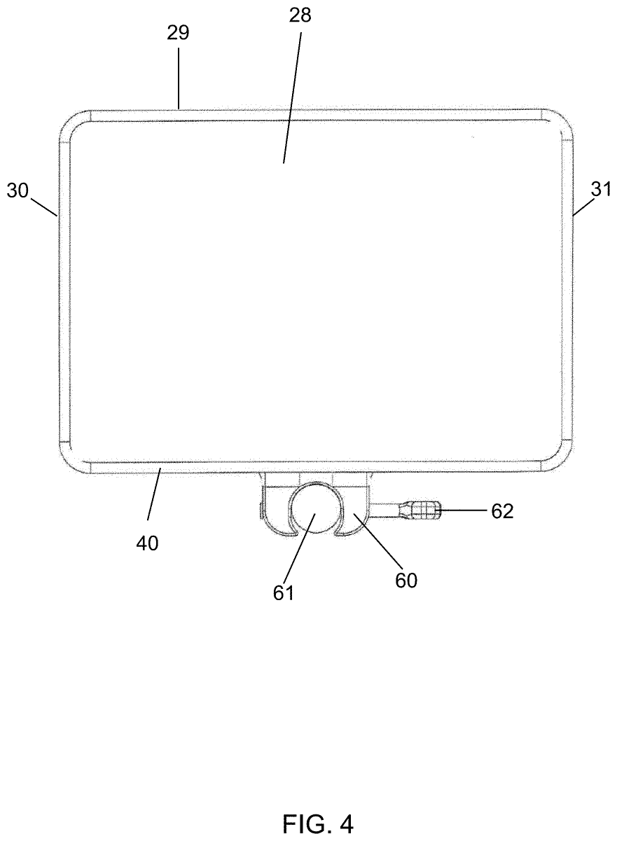

FIG. 5 is a front view of the surgical sponge counter bag dispenser for the surgical sponge accounting system according to one embodiment.

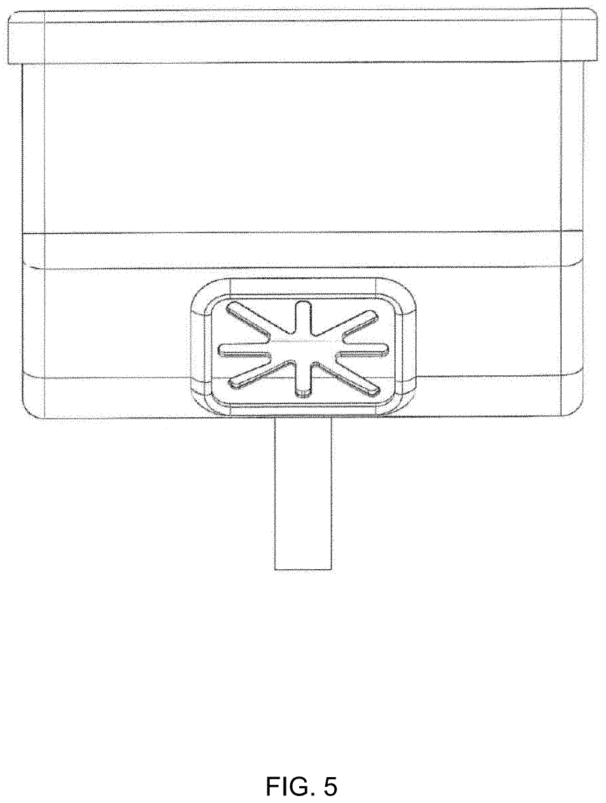

FIG. 6 is a side view surgical sponge counter bag dispenser for the surgical sponge accounting system according to an alternative embodiment.

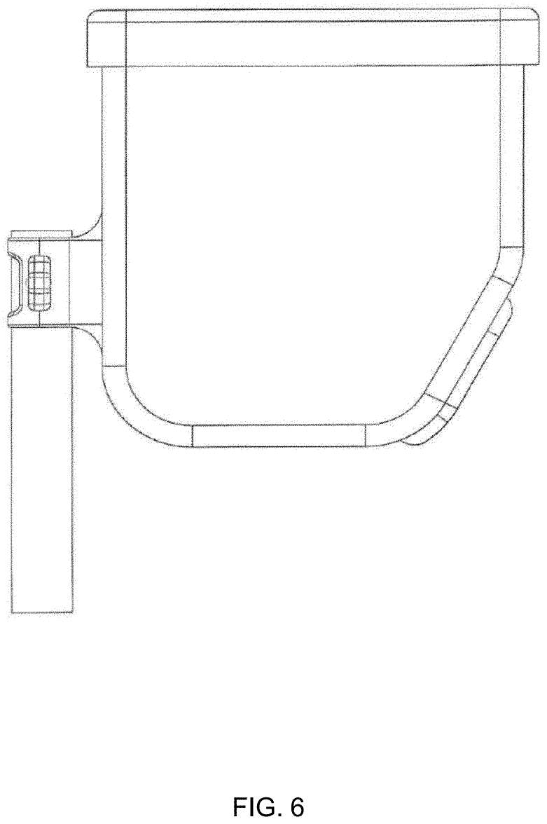

FIG. 7 is a rear perspective view surgical sponge counter bag dispenser for the surgical sponge accounting system according to an alternative embodiment.

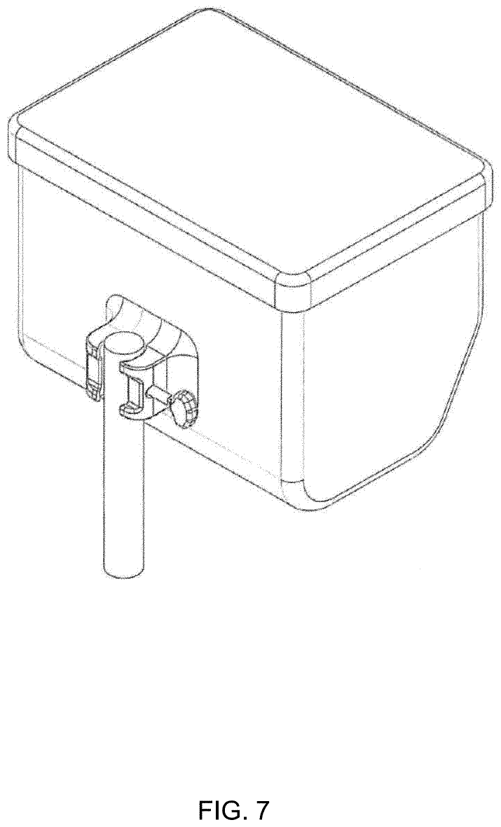

FIG. 8 is a back view surgical sponge counter bag dispenser for the surgical sponge accounting system according to an alternative embodiment.

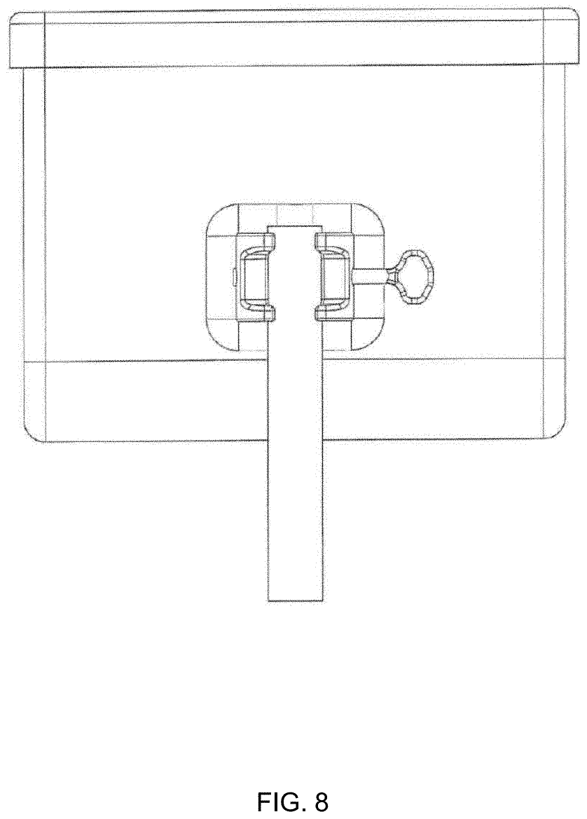

FIG. 9 is a front view surgical sponge counter bag dispenser for the surgical sponge accounting system according to an alternative embodiment.

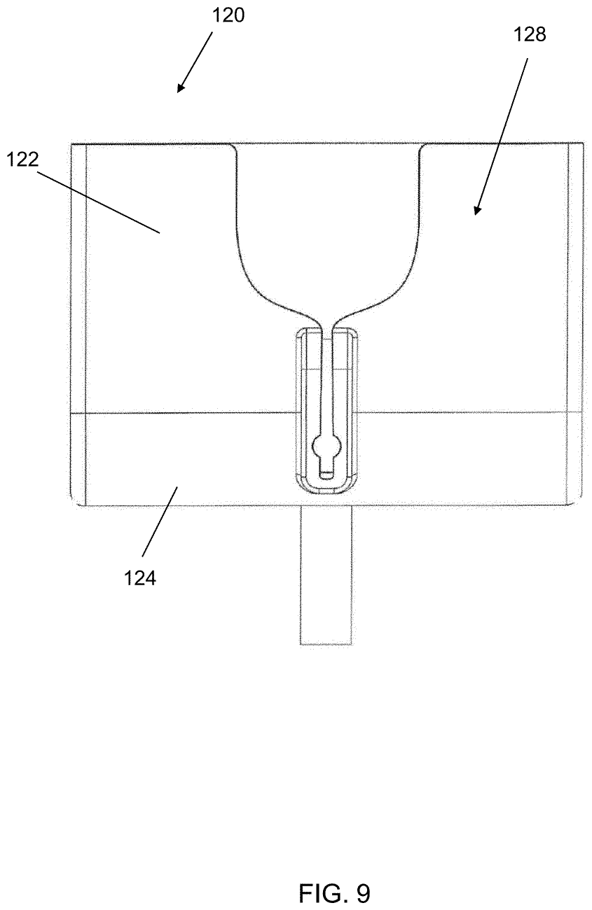

FIG. 10 is a side view surgical sponge counter bag dispenser for the surgical sponge accounting system according to an alternative embodiment.

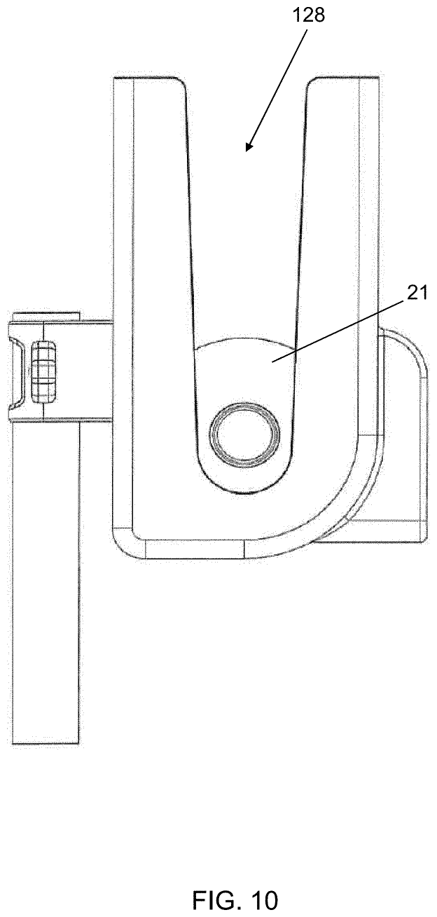

FIG. 11 is a rear perspective view surgical sponge counter bag dispenser for the surgical sponge accounting system according to an alternative embodiment.

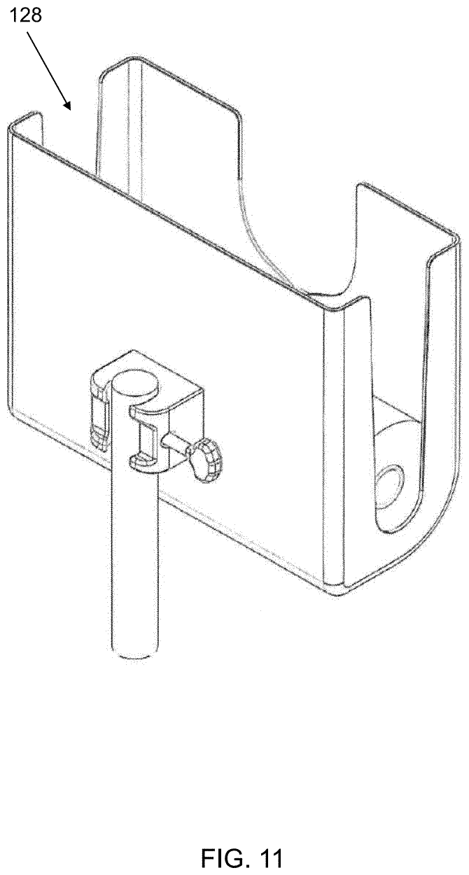

FIG. 12 is a front view surgical sponge counter bag dispenser for the surgical sponge accounting system according to an alternative embodiment.

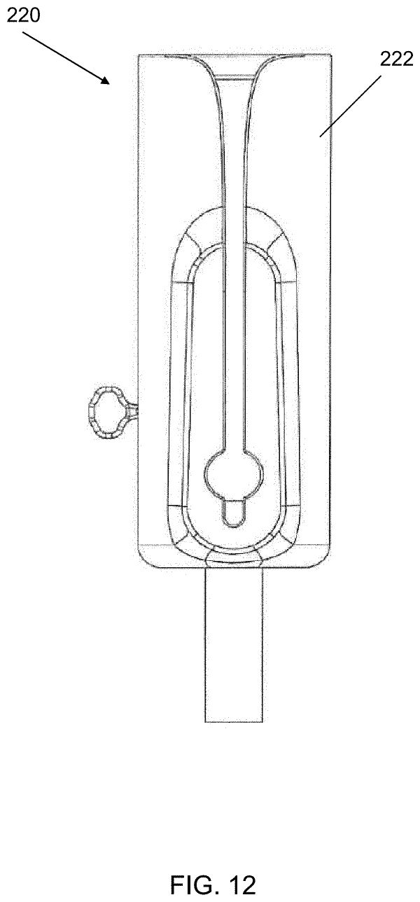

FIG. 13 is a side view surgical sponge counter bag dispenser for the surgical sponge accounting system according to an alternative embodiment.

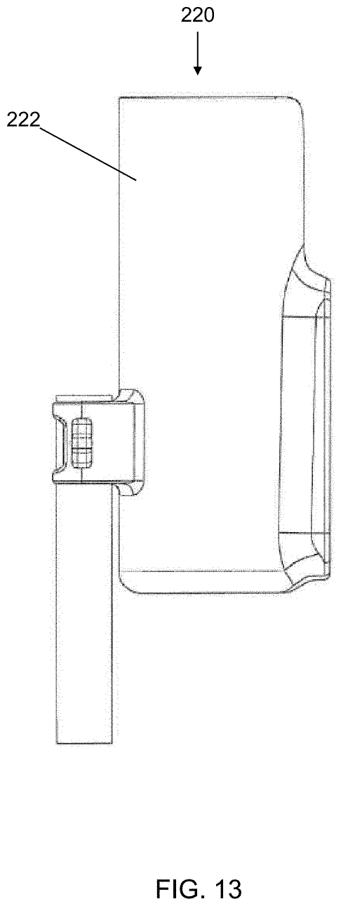

FIG. 14 is a rear perspective view surgical sponge counter bag dispenser for the surgical sponge accounting system according to an alternative embodiment.

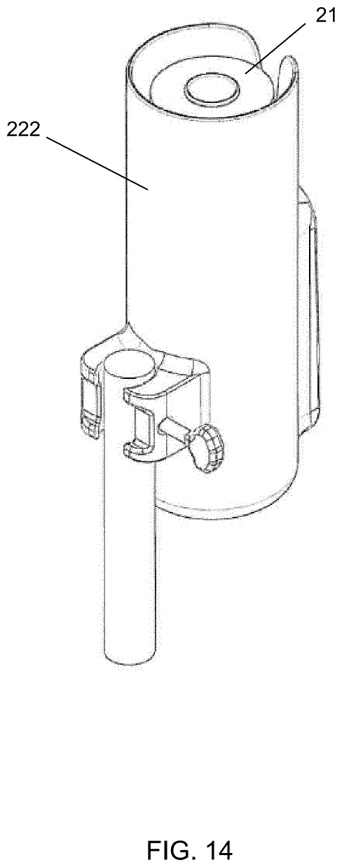

FIG. 15 is a top view surgical sponge counter bag dispenser for the surgical sponge accounting system according to an alternative embodiment.

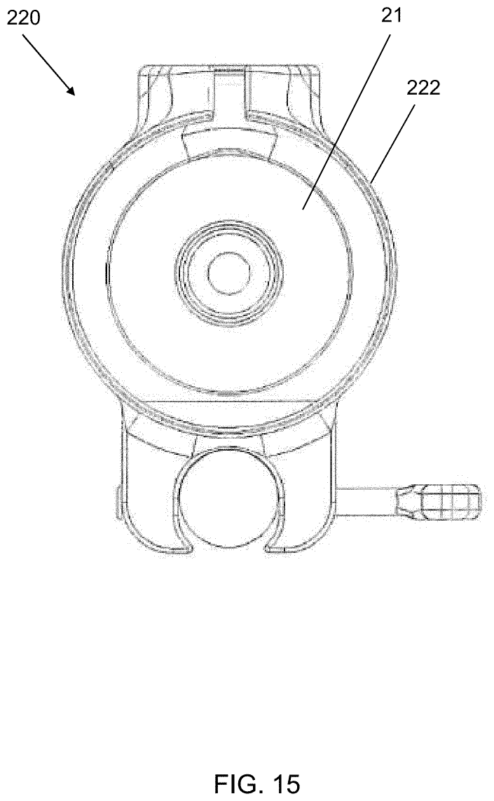

FIG. 16 is front view surgical sponge counter system.

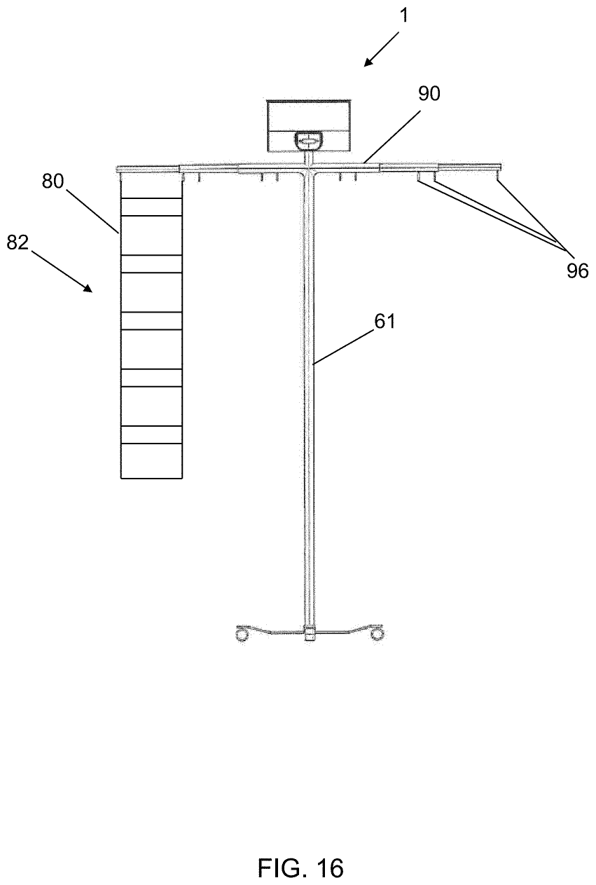

FIG. 17 is a rear-view surgical sponge counter system.

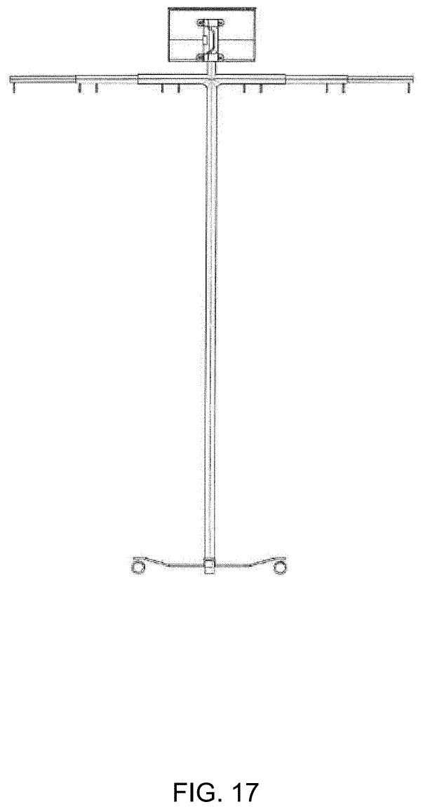

FIG. 18 is a top view surgical sponge counter system.

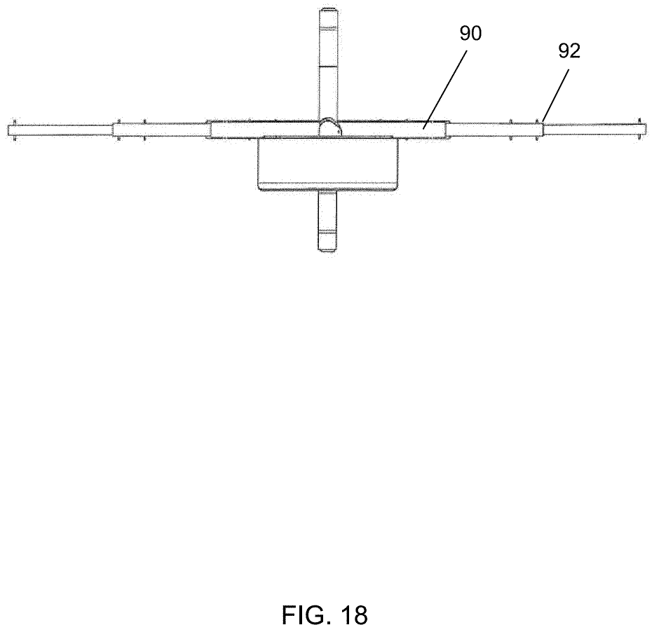

FIG. 19 is a side view surgical sponge counter system.

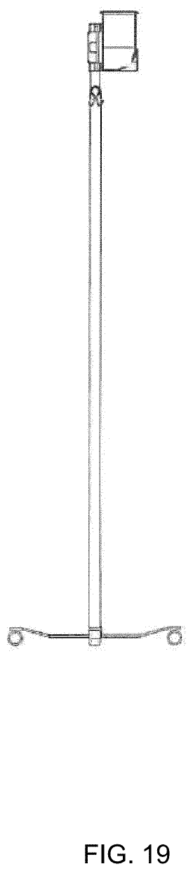

FIG. 20 is view of the hooks for the surgical sponge counter system.

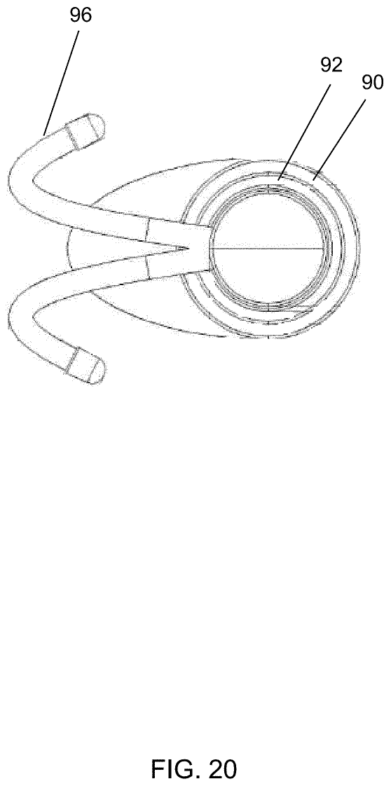

### DETAILED DESCRIPTION

The present invention is directed to a surgical sponge accounting system and the method of use. The invention provides a novel surgical sponge counter bag dispenser with a used surgical sponge holder for displaying the surgical sponge counter bags. The overall device creates a system that dispenses surgical sponge counter bags for use in accounting for surgical sponges, the device further provides a counting station for the organization, placement and accounting of used surgical sponges (or other surgical items) within the surgical sponge counter bags and a visible method for accounting for the whereabouts of the used surgical sponges (or other surgical items) during and at the conclusion of the surgical procedure, whereby creating a systematic process to account for surgical sponges and other items after a surgical procedure. With reference to the figures, the invention is to a novel surgical sponge accounting system that includes various embodied principles and concepts that create a system that offers a simple, efficient and reliable method for safely accounting for used surgical sponges and/or other surgical items during and at the conclusion of a surgical procedure.

As illustrated in the figures provided, the surgical sponge accounting device 1 and system comprises a housing member 20 with a perimeter wall 22 to create four wall sides. The surgical sponge system can further comprise a bottom wall member 24 that connects to the perimeter wall 22. In an alternative embodiment the housing can include any number of perimeter walls to create an interior area 28. The perimeter walls can further be configured to attach to a lower viewing assembly 26, instead of a bottom wall, that includes a plurality of walls with a bottom surface configured in a shape to accommodate a roll of surgical sponge bags. The perimeter walls 22 include a first lateral wall 29, a second lateral wall 30, a third lateral wall 31 and a fourth or back wall 40. The perimeter walls 22 are configured as a single piece of material or as separate walls fixedly attached with the bottom wall either created as a single piece with the perimeter walls or as a separate wall fixedly attached. The perimeter walls and viewing assembly can also be configured as a single piece of material or as separate walls fixedly attached.

In another embodiment the housing member 120 can include perimeter walls 122 and a bottom wall 124 with the perimeter walls configured with opening space(s) on the walls and an open top that allows for the roll of surgical sponge bags 21 to be placed within the internal space 128 of the housing member 120. The open cut-out or space of the perimeter walls allows for a user to visualize the roll of surgical sponge bags to monitor if bags remain. In another embodiment the housing member 220 can be configured with one perimeter wall 222 in a circular shape that allows for a roll of surgical sponge bags 21 to be placed in an up-right position for the delivery of the bags to a user.

The housing further includes a lid 42. The lid is coupled in communication in either a fixedly or removable connection to the back wall 40 or one wall of the perimeter walls 29 or 31. The lid has a top surface and bottom surface, with a defined edge. The edge can be configured to allow for easy grip or to access the internal space within the housing member 20. The lid is configured to be positonable to cover the open top of the housing. The bottom surface with the edge of the lid is designed to allow for a lip that extends past the edge of the perimeter walls 22. The extension past the perimeter walls can be on one, two, three or all four of the lateral walls of the housing. The lid further includes a hinge 44 or other connection mechanism for connecting the lid to the housing member. The lid hinge may be positioned between the top edge of the lid and an upper edge of the back wall or a wall of the housing. The lid hinge creates a pivot point between the lid and the housing (preferably on the back wall) to allow for the transition between on open position and a closed position for the lid. The lid hinge can also be configured to hold the lid in an open or closed position as desired by the user. Closing the lid creates a closed interior within the housing where the roll of surgical sponge bags can be placed for dispensing to a user. In other embodiments the lid can be connected to the housing unit by any method known within the industry, to allow for opening and closing of the system. The lid can further include a locking or securing mechanism to hold the lid in a closed position. Any locking or securing mechanism known in the industry can be used.

The housing member can further include a lower viewing assembly that includes a viewing configuration to allow a user to monitor the roll of surgical sponge bags and ensure there is an adequate amount of bags left on the roll. The viewing assembly can be composed of any material that will allow a user to view the internal space within the housing member, this can include but is not limited to any clear material such as plastic or other materials used within the industry. The lower assembly is configured with material that allow a user to view within the internal area of the housing. The lower viewing assembly is configured with a plurality of walls to further extend the space within the housing assembly. In one embodiment, the walls will include a back wall and lateral side walls with a front wall configured opposite the back wall. The front wall configuration can include a circular curve or in an alternative a slant to allow for the accommodation for the roll of surgical sponge bags. The curved configuration of the front panel of the lower viewing assembly will further correspond to lateral walls on the lower viewing assembly that are also curved to accommodate the desired shape to accommodate the roll of surgical sponge bags 21. In alternate embodiments, the lower viewing assembly can be of any shape that will accommodate a roll of surgical sponge bags and allow for a user to view the roll of bags while internal within the housing.

The housing further includes a dispensing opening 50 configured to allow for the release of surgical sponge bag(s) from within the housing unit. In one embodiment the dispensing opening is on the viewing assembly and the opening is configured to allow for the surgical sponge bags that are within a roll to feed out through the dispensing opening. In another embodiment, the dispensing opening can include a slit 52 or other minimized opening that is configured to allow for a small space where the surgical sponge bags are released to the outside of the housing. In another embodiment the dispensing opening is on a perimeter wall of the housing to allow for bags to be dispensed to a user. The dispensing opening can be an opening, a slit, a cut-out or any other means known in the industry that allows for the passage of the surgical sponge bags that are in a roll configuration to be dispensed and released to outside of the housing member. In another embodiment the dispensing opening 54 can include material that is flexible and overlaps itself to create an amount of friction on the surgical sponge bags as it is released from the internal space of the housing. The material creates a barrier between the inside area of the housing unit and the outside of the housing unit.

The housing includes a connection mechanism or clamp 60 designed to connect with the outer surface of the back wall of the housing. The connection mechanism is configured to allow for the connection of the housing unit to the top end of an IV pole 61. In additional embodiments the housing can include a connection mechanism that allows for the connection to a wall member, a ceiling member or any other similar apparatus that allows the housing unit to be located in a position near the patient while in use but stored out of the way when not in use. The connection mechanism in one embodiment is designed as a clamp fixedly secured to the housing unit that can then be locked into place on an IV pole with a tightening clamp 62 as typically found within the healthcare industry for use on IV poles. The clamp allows for the easy release or connection of the housing unit to the IV pole. In other embodiments the housing can include any connection mechanism known in the industry that is able to connect the housing to an IV pole. In a further embodiment the housing can include any connection mechanism that allows the unit to be attached to an object, wall or ceiling to hold the housing in place during a procedure as well as out of the way for storage after a procedure.

The surgical sponge accounting system can accommodate a plurality of surgical sponge bags 80 that have been unrolled for use. The surgical sponge bags are typically configured as hanging panels 82. The surgical sponge counter bags are typical bags used within the industry. Each of the hanging panels has an upper edge and a lower edge. The hanging panels are coupled together to form a roll configured to fit within the confines of the internal space of the housing. The hanging panels 82 configured within a roll are then allowed to rotate outwardly through the dispensing opening. The surgical sponge roll 21 is configured to include perforations attached together and evenly spaced throughout the roll. These perforations created lower and upper edges for each potential hanging panel and are configured to permit the hanging panel to be detached from the roll. In one embodiment the configuration of the perforations will include a hanging panel that has two (2) columns and five (5) rows, for a total of 10 pouches to hold used surgical sponge. In other embodiments number of columns are twenty and the number of rows are fifty. In alternative embodiments the number of columns and rows will be configured to meet the needs of any surgical procedure and based on the space available within a surgical room. The hanging panels 82 of the surgical sponge bags 80 with the columns and rows configuration allows a user to view each pouch and further the viewing of a used surgical sponge positioned within the interior space of the associated pouch.

The surgical sponge accounting system further includes an organization attachment member 90 configured for connecting in conjunction with the housing to an IV pole 61. The organization attachment works in communication with the dispenser housing unit to create the overall device and complete system. In one embodiment the organization attachment member and housing member are configured as a single integrated member. In another embodiment the organization attachment member and housing member are separate but work in communication to create the complete sponge accounting system. In additional embodiments the housing member can work in conjunction with other organization attachment members currently used to create the overall sponge accounting system. The organization member is configured in a t-shape design with a first and second pole extension side (92 and 94). The first extension pole and second extension pole sides are configured with a plurality of hooks 96. The hooks are configured to correspond to the hanging holes positioned in each of the hanging panels 82. Each of the hanging holes on the hanging panels are positioned proximate the upper edge of the associate hanging panel. Each of the hanging holes of the associated hanging panel is spaced apart a distance equal to the distance between a pair of hooks on the organization member. The first extension pole is configured to extend or contract dependent on the number of hanging panels to be hung on the hooks. In one embodiment the organization attachment first and second extension pole will include three (3) pairs of hooks to accommodate three (3) hanging panels on each side for a total of six (6) hanging panels. In another embodiment the extension pole will include several hooks that will accommodate the number of panels that can be used during a procedure. The number will vary based on use and is not limited to a specific number of hooks to accommodate the hanging panels. The spacing of the hooks will be based on the hanging panels used during the procedure to hold the sponges. The configuration of the extension pole sides allow for the accounting attachment member to be expanded or contracted dependent on the total number of hanging panel needed to accommodate the used surgical sponges, from a procedure, to be placed in the pouches/compartments found on a hanging panel. In a further embodiment the organization attachment member will have any connection mechanism like the housing member that allows attachment to an IV pole or to an object, wall or ceiling to hold accounting attachment member in place during a procedure as well as out of the way for storage after a procedure.

The method for using the surgical sponge accounting system is important to limit or eliminate RSI events. To begin a medical professional performs an initial count of soft sponges, sharps and any other instruments that may be used during a procedure. To begin the sterile sponges, after counted, are placed on the sterile field, for use throughout the surgery. During the surgery the medical profession may add additional sterile surgical sponges to the sterile field, each time additional sponges are added a new total count is recorded for the surgical items. As the sterile sponges are used they are discarded into a kick-bucket by the surgical technician/scrub RN to be later separated by the un-scrubbed circulating RN/medical professional and placed in the sponge counter bags that are dispensed from the surgical sponge accounting system and are hung on the surgical sponge organizing member of the surgical sponge accounting system. Throughout a surgery additional sponges may be required and with the use of the surgical sponge accounting system each additional sponge can be efficiently accounted for. After surgeries where an incision is made the medical professional will also perform a closing and final count for surgical items where all countable items are accounted for before completely closing the incision of the patients. When counting sponges by utilizing the surgical sponge organizing system and sponge counter bags, the unscrubbed RN will make each bag fully visible to the scrubbed in person at the sterile field. The surgical count whether it be initial, closing or final will involve both the surgical technician/scrub RN and the un-scrubbed circulating RN, this allows for both parties to visually see and account for the surgical items being counted and keep track of each of these counts according to each facilities count policy.

Thus, there has been shown and described a surgical sponge accounting system and method of using the same. It is apparent to those skilled in the art, however, that many changes, variations, modifications, and other uses and applications for the system are possible, and such changes, variations, modifications, and other uses and application, which do not depart from the spirit and scope of the invention, are deemed to be covered by the invention, which is limited only by the claims which follow.

What is claimed is:

1. A surgical sponge accounting system comprises:
a housing unit, including a viewing assembly,
a dispensing opening;
and a organization attachment member configure with telescoping sides and a plurality of hooks on the telescoping sides to accommodate a hanging panel for use with used surgical sponges.

2. The surgical sponge accounting system of claim 1, wherein the housing unit includes a plurality of walls that further includes a front wall and a rear wall.

3. The surgical sponge accounting system of claim 1, further includes a lid connected to the housing unit.

4. The surgical sponge accounting system of claim 1, wherein the viewing assembly includes a plurality of walls with a front wall configured in a circular curve to accommodate a roll of surgical sponge bags.

5. The surgical sponge accounting system of claim 1, the dispensing opening further includes a flexible material that creates an overlap open slit over the dispensing opening.

6. The surgical sponge accounting system of claim 1, further includes a clamp on the housing unit.

7. The surgical sponge accounting system of claim 6, wherein the clamp is configured on the back wall of the housing unit.

8. The surgical sponge accounting system of claim 7, wherein the clamp further includes a tightening mechanism to allow for secured connection to an IV pole.

9. The surgical sponge accounting system of claim 1, further includes a mounting rod wherein the mounting rod is connected to the housing unit.

10. The surgical sponge accounting system of claim 1, wherein the organization attachment is configured in a t-shape design with a first and a second pole extension.

11. The surgical sponge accounting system of claim 1, wherein the organization attachment is configured as fixedly attached to the housing member.

12. The surgical sponge accounting system of claim 1, wherein the organization attachment is configured as removably attached to the housing member.

13. The surgical sponge accounting system of claim 10, wherein the first pole extension and the second pole extension include a telescoping configuration where the first and the second pole extension can be extended or collapsed to allow for holding a sponge bag.

14. The surgical sponge accounting system of claim 1, further includes a plurality of hooks on the first pole extension and the second pole extension.

15. The surgical sponge accounting system of claim 1, further includes a hanging panel that is a used sponge bag that is supported on the plurality of hooks, wherein the sponge bag includes pockets configured to receive and display surgical sponges.

16. The method for accounting for surgical sponges including storing, displaying and counting use surgical sponges on a surgical sponge accounting system, wherein the surgical sponge accounting system includes a housing unit, including a viewing assembly, a dispensing opening, a clamp to connect the housing unit to an IV pole; and a organization attachment member configure with telescoping sides that includes a first pole extension and a second pole extension, and a plurality of hooks on the telescoping sides to accommodate a hanging panel of sponge bags for use with used surgical sponges.

17. The method for accounting for surgical sponges of claim 16, further including expanding the first pole extension or the second pole extension to accommodate additional hanging panels of sponge bags to fill with used surgical sponges.
